(19) United States
(12) Patent Application Publication
(10) Pub. No.: US 2006/0036248 A1
(43) Pub. Date: Feb. 16, 2006

(54) Fixation elements

(76) Inventors: Joseph M. Ferrante, Bartlett, TN (US); N. Kelley Grusin, Germantown, TN (US); Anthony James, Bartlett, TN (US)

Correspondence Address: CHIEF PATENT COUNSEL;SMITH & NEPHEW, INC., 1450 BROOKS ROAD, MEMPHIS, TN, 38116, US

(21) Appl. No.: 11/173,532

(22) Filed: Jul. 1, 2005

Related U.S. Application Data
(60) Provisional application No. 60/584,557, filed on Jul. 1, 2004.

(51) Int. Cl. A61B 17/56

(52) U.S. Cl. 606/64

(57) ABSTRACT

The present invention provides a device for treating fractures of a bone and methods for treating a facture, particularly fractures of the femur, that uses an intramedullary nail or a bone plate and a sliding compression fixation element. Certain features of various fixation elements described herein lessen the rotational forces applied during implantation and/or lessen the amount of bone that needs to be removed during placement of the sliding compression screw.

This application claims priority to U.S. Provisional Application Ser. No. 60/584,557, filed Jul. 1, 2004, titled “Intramedullary Nail Fixation Elements,” the entire contents of which is hereby incorporated by reference.

### FIELD OF THE INVENTION

The present invention relates to devices used to treat bone fractures, and particularly relates to compression system fixation elements for securing fractured portions of a femoral head, neck or shaft across a fracture line.

### BACKGROUND

The number of hip fractures occurring every year continues to increase. Most hip fractures happen in elderly patients who slip and fall or who have diseases that weaken the bone. Hip fractures may also occur in younger patients due to high-energy physical trauma, such as motor vehicle accidents and the like. Intertrochanteric and femoral neck fractures are the most common types of proximal fractures, although subtrochanteric and greater trochanter fractures also occur with some frequency. For almost all types of fractures, however, surgery is typically required to avoid further displacement and alleviate pain.

A primary goal of hip fracture treatment surgery is to stabilize the fracture site and allow the fragmented bone to heal. One type of implant that has been used to treat proximal femoral factures is a compression plate having a barrel member, a lag screw, and a compression screw. With this type of implant, a compression plate is secured to the exterior of a femur and the barrel member is inserted into a pre-drilled hole in the direction of the femoral head. The lag screw, which has a threaded end and a smooth portion, is inserted through the barrel member so that it extends across the break, and the threaded portion extends into the femoral head. A compression screw connects the lag screw to the plate. The fracture is reduced (or compressed) by adjusting the tension of the compression screw, and the smooth portion of the lag screw is allowed to slide through the barrel member to permit adjustment of the compression screw.

One problem with this type of implant is that it can cause rotation at the fracture site. That is, the rotation of the lag screw as it is being twisted into the femoral head can cause the head to rotate, causing misalignment, particularly because the femoral head (or other bone fragment to be reduced) is separated. Accordingly, it is desirable to provide a lag screw-type system that provides secure attachment into the bone, but that does not cause rotation of the bone fragment during insertion and placement of the screw.

Another problem with the bone plate system is that the incision required to place the implant must be equal to the length of the plate. Accordingly, many systems now use an intramedullary nail, as described below.

Moreover, osteogenic patients may not have adequate bone mass (or the remaining bone that is present may be insufficient) for the lag screw to achieve sufficient purchase. Again, it is desirable to provide a compression system that securely attaches the lag screw to the bone, regardless of whether the patient's bone quality is poor.

Another type of implant that may be used to treat hip fractures is an intramedullary nail (or rod) and compression screw system. With this implant, an intramedullary nail is placed into a patient's femoral canal and a sliding lag screw, again having a threaded end a smooth end, slides through the nail for improved compression. The threaded end of the screw engages bone on one side of the fracture, and the smooth portion of the screw cooperates with the nail on the opposite side of the fracture. As the patient begins to bear weight on the fractured site, the bone fragments are further compressed together.

However, as with the plate system, the nail and compression screw system may also cause rotation of the femoral head during placement of the lag screw. It is thus desirable to provide a system that can eliminate this rotation problem.

Further implants used to treat hip fractures may include the use of two or more screws to stabilize the fracture at more than one location. This can help prevent some of the rotation that may occur during the placement of a single screw. Two or more screws may also be required in instances where multiple fractures of the same bone or area need to be treated.

Some systems are provided that use talons, tangs, or moly bolts that extend out from a lag screw to grab bone. Although these systems may achieve good bone fixation, they still can cause rotation of the bone fragment (for example, the femoral head) during placement of the lag screw (i.e., as the surgeon twists the screw) due to the threads or blades at the tip of the screw that initially engage the bone.

Another challenge that is sometimes encountered with some hip fracture compression treatments is that the reaming of the hole to receive lag screw may require removal of more bone than desired. This is because the surgeon needs to ream the portion of the bone fragment closest to the nail or plate to be large enough so that it will receive the smooth portion of the screw that will slide in relation to and cooperate with the nail and another portion of the bone fragment to receive the threads of the lag screw. The first reamed hole is slightly larger than the outer diameter of the screw threads to (a) allow the screw threads to pass through the hole and engage the bone of the other side of the fracture but to also (b) allow the smooth portion of the screw to slide and be compressed against the nail or plate. Accordingly, it is also desirable to provide a system that can eliminate or reduce the removal of excess bone needed for lag screw placement, particularly because the bone in many hip fracture patients is already comprised or weak.

### SUMMARY

The present invention provides a device for treating fractures of a bone and methods for treating a facture, particularly fractures of the femur, that uses an intramedullary nail or a bone plate or other osteosynthetic device and a sliding compression fixation element. Certain features of various fixation elements described herein lessen the rotational forces applied during implantation and/or lessen the amount of bone that needs to be removed during placement of the sliding compression screw.

One embodiment of a fixation element according to certain embodiments of the invention comprises a shaft having a bone engaging end portion and a driving end portion, the bone engaging end portion having a series of substantially straight flutes for engaging bone, the shaft having one or more protruding elements adapted to be deployed to engage bone and to secure the fixation element in place during use, and the driving end adapted to receive a tool for deploying or retracting the one or more protruding elements.

Other embodiments of the invention comprise a shaft comprising threads having a substantially flat crest along a substantial length of the shaft, and a bone engaging portion comprising threads having a narrow crest for engaging bone.

Further embodiments of the invention comprise methods of placing the fixation elements described herein, the methods comprising inserting an osteosynthetic device having at least one opening through the osteosynthetic device into the patient's femoral canal or secured onto the side of a patient's femur, inserting a fixation element into the opening of the osteosynthetic device and into the patient's femoral head, such that the fixation element crosses the fracture, deploying one or more protruding elements of the fixation element (if provided) to engage the femoral head and secure the fixation element from axial and rotational movement; and securing the fracture to achieve fixation.

### BRIEF DESCRIPTION OF THE DRAWINGS

FIG. 1 shows a side perspective view of a fixation element according to one embodiment of the invention.

FIG. 2 shows a cross sectional view of the fixation element of FIG. 1.

FIG. 3 shows a perspective view of a fixation element according to another embodiment of the invention.

FIG. 4 shows a perspective view of a fixation element according to a further embodiment of the invention.

FIG. 5 shows a side plan view of a fixation element according to a further embodiment of the invention.

FIG. 6 shows a cross sectional view of the fixation element of FIG. 5.

FIG. 7 shows a perspective view of the fixation element of FIG. 3 in cooperation with an osteosynthetic device.

FIG. 8 shows a perspective view of the fixation element of FIGS. 5-6 in cooperation with an osteosynthetic device.

FIG. 9 shows a perspective view of a tool for use in connection with certain embodiments of the invention.

### DETAILED DESCRIPTION OF THE DRAWINGS

The present invention relates to a fracture treatment system 10 that includes an osteosynthetic device 12 (which is shown as an intramedullary nail, but it should be understood that a bone plate or any other osteosynthetic device may be used in connection with this invention) and a fixation element 20, 70. The device 10 is particularly useful for the treatment of long bone fractures, predominantly for the treatment of fractures of the proximal femur. (For the purposes of this description, the fixation elements will be described in relation to an intramedullary nail and for use to treat a femoral fracture. However, it should be understood that they may also be used in connection with bone plates or any other stabilizing device for repairing or securing bone fractures or other conditions requiring the use of a fixation structure in any other part of the body, such as the shoulder, the knee, and so forth.)

The fracture treatment system 10 and its components may be made of any suitable strong, biocompatible material, such as stainless steel, titanium, cobalt-chrome or any other material having sufficient strength and biocompatibility.

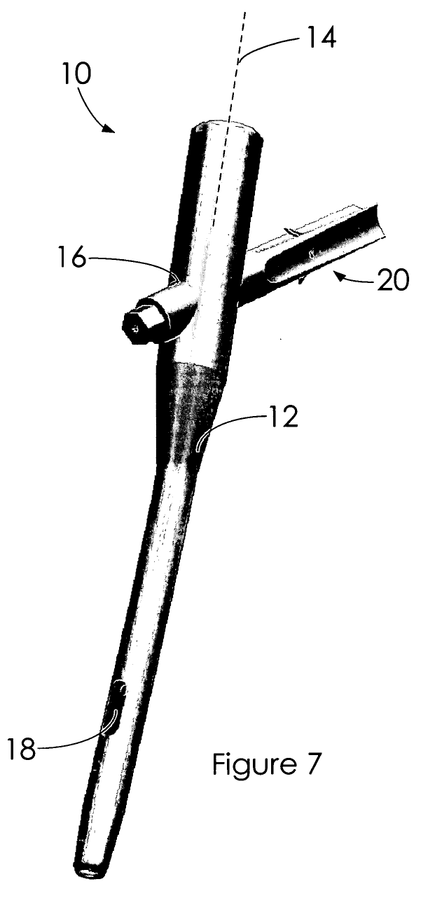

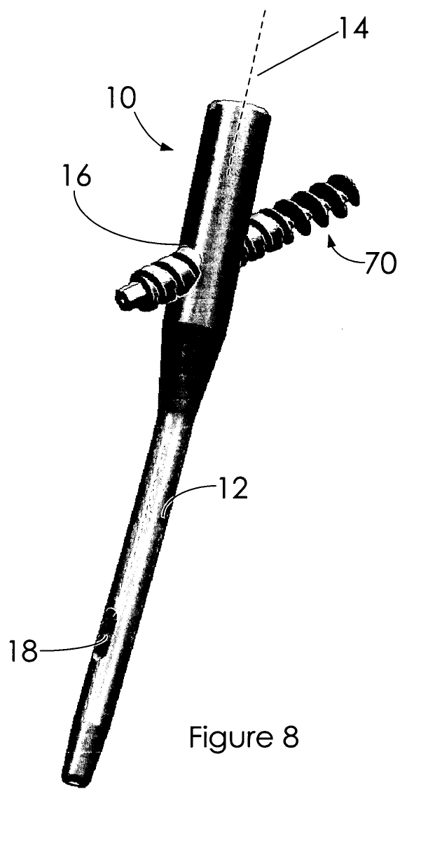

As shown in FIGS. 7 and 8, the osteosynthetic device 12 of certain embodiments of the invention has a longitudinal axis 14 that may either be cannulated or may have a closed cross section. In some embodiments, the longitudinal axis 14 may be curved to follow the natural curve of the femur. The device 12 has at least one pair of holes 16 arranged co-axially and preferably extending in a transverse direction across the longitudinal axis 14 of the device 12, such that the holes 16 are adapted to slidingly receive a fixation element 20, 70 that is adapted to be inserted through the osteosynthetic device 12. (The osteosynthetic device 12 may further include additional anchoring receiving holes 18 that are adapted to receive a nail, screw or bolt to secure the rod within the intramedullary canal of the femur.) An exemplary device that may be used in connection with any of the fixation elements 20 described below is shown and described in U.S. Pat. No. 4,827,917 to Brumfield, the entire contents of which are incorporated here by this reference.

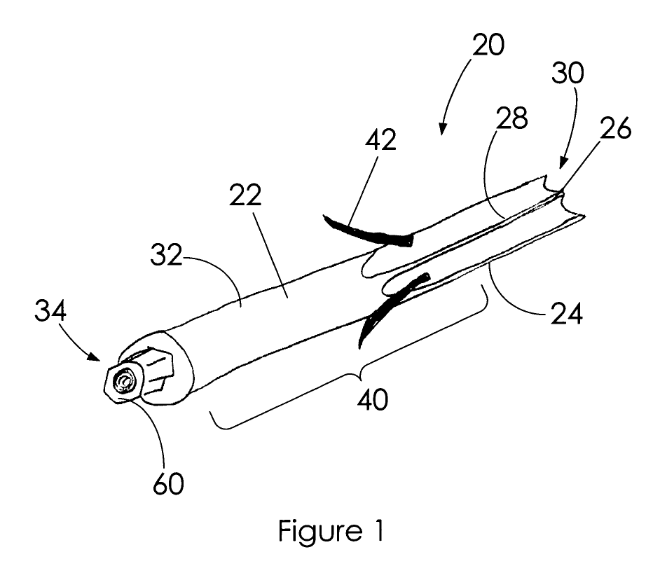

As shown in FIGS. 1-4, one type of fixation element 20 according to certain aspects of this invention features a shaft 22 with substantially straight flutes 24. (“Substantially straight” is used in this document to mean that the flutes do not twist around the end of the shaft, however, slight variations (e.g., manufacturing tolerances or slightly angled flutes) that result in flutes not being perfectly straight are still considered within the scope of this invention.) Substantially straight flutes 24 allow the fixation element 20 to be driven across a fracture site such that the flutes 24 engage bone without the element 20 being twisted. Flutes 24 do not require the typical rotation motion that screw threads require for engaging bone, and accordingly, the risk of rotating the femoral head out of alignment is greatly lessened.

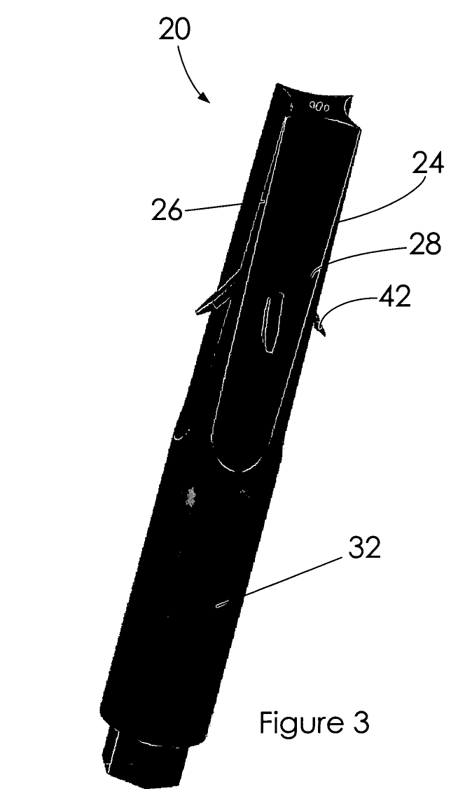

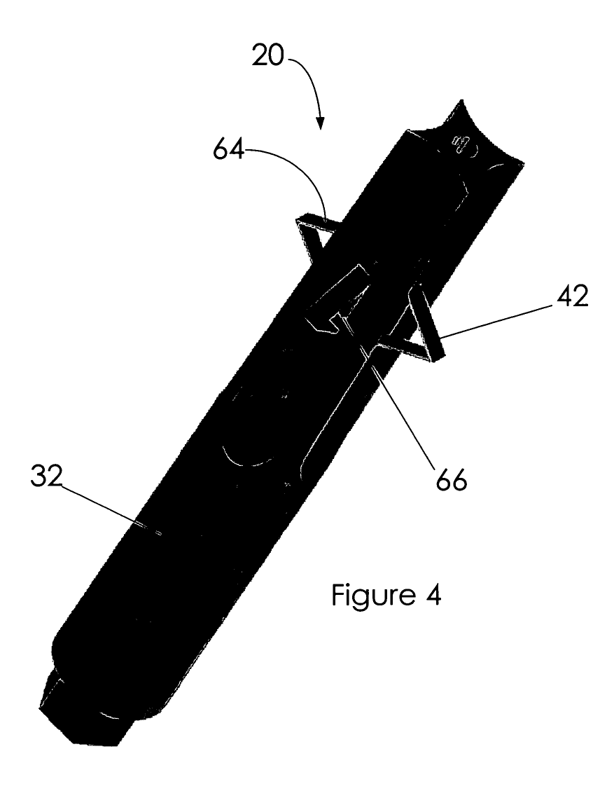

Flutes 24 may be provided in any shape and size. The top (or apex) 26 of each flute may be rounded, square, triangular or pointed, oblong, or any other desired shape. FIG. 1 shows flutes 24 that have a square apex 26 and substantially parallel sides 28. FIG. 3 shows flutes 24 that have a pointed apex and sides 28 that are tapered in a longitudinal direction as well as tapered inwardly. As shown, the sides 28 of each flute may be parallel or tapered or any other desired configuration. Flutes 24 preferably extend to the bone engaging end 30 of shaft 22, although if desired, the bone engaging end 30 of shaft 22 may have a point, a self-tapping end, or other shape that will facilitate passage through and engagement with bone. The outer diameter 40 of shaft 22 (formed in part by flutes 24) may be circular, square, oblong, rectangular, or any other desired configuration. Additionally, in some embodiments, the bone engaging end 30 of shaft 22 has a slightly smaller diameter than the diameter at the other end (the driving end 34), providing a wedge-type shaped fixation element 20 that can be more fully seated in bone. An example of a smaller diameter that forms a wedge is shown in FIGS. 3 and 4.

In some embodiments, substantially straight flutes 24 extend along the entire distance of shaft 22. In other embodiments, flutes 24 are only provided along a portion of shaft 22, for example, the portion that engages bone. In this instance, the other part of shaft 22, the part that cooperates with the osteosynthetic device 12, may be a substantially smooth portion 32. (“Substantially smooth” is intended to refer to a smooth portion that may have slight imperfections that would otherwise prevent the surface from being considered perfectly smooth. Such surfaces are still considered within the scope of this invention.) If provided, the substantially smooth portion 32 is sized to be received through holes 16 of osteosynthetic device 12 (which again, is shown as an intramedullary nail, but may be a bone plate or any other device adapted to secure a fracture). Again, the outer diameter 40 of the substantially smooth portion 32 may be circular, square, oblong, rectangular, or any other desired configuration, as long as it is allowed to slide with respect to holes 16. (Note that although holes 16 will typically be circular, they may also be provided in any desired shape.) In use, substantially smooth portion 32 allows the fixation element 20 to be used for sliding compression of the fracture.

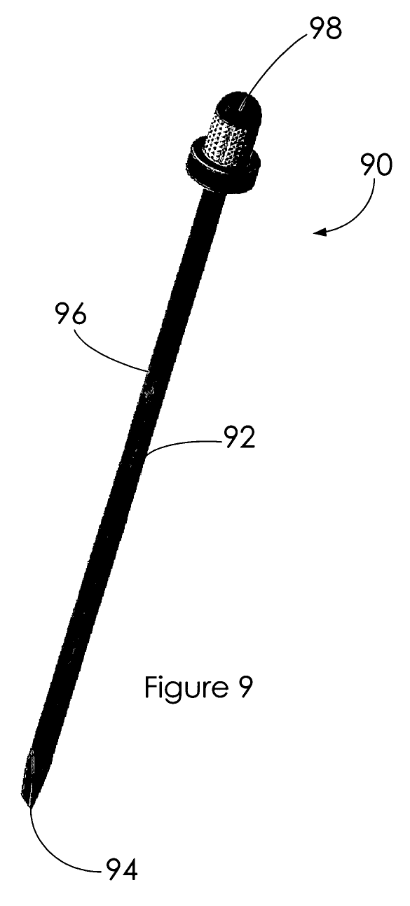

Drive connector 60 is located at the driving end 34 (the end opposite the bone engaging end 30 of shaft where flutes 24 are located) of fixation element 20. Drive connector 60 is adapted to be attached to a driver that is used to place fixation element 20. Driver may or may not be associated with the tool 90, shown in FIG. 9, that is used to deploy protruding elements 42. In some embodiments, a multi-sided protrusion or inset, such as a hexagonally shaped inset at the drive connector 60 permits insertion of a suitable driver for placement of fixation element 20. In certain embodiments, the driver is adapted to drive the fixation element 20 straight into the bone, as opposed to the typical drivers that are used to twist a screw into bone.

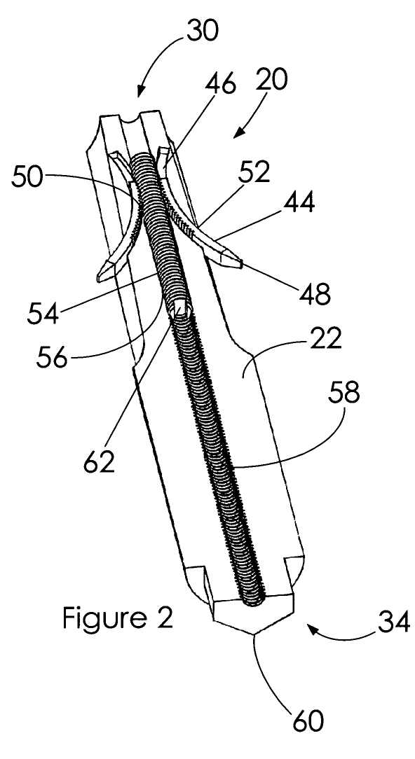

However, because substantially straight flutes 24 are not twisted into the bone, fixation element 20 runs the risk of pulling out of the bone or advancing too far into the bone if no other securing mechanism is used. Accordingly, shaft also has deployable and retractable protruding elements 42, various embodiments of which are shown in FIGS. 2 and 4. When protruding elements 42 are deployed, they engage the bone of the femoral head (or other fracture site) to increase purchase (axial fixation) and rotational stability of fixation element 20. “Protruding element” is being used in this specification to refer to any member that extends out from fixation element (preferably in a non-parallel fashion) even if only slightly, such that it can engage bone and stabilize fixation element.

Protruding elements 42 are deployable and retractable, such that they remain retracted during placement of fixation element 20 and are deployed once fixation element 20 is in place. If fixation element ever needs to be removed, the protruding elements 42 may be retracted.

The shaft 22 of fixation element 20 is preferably cannulated or has an opening 58 that runs through the shaft 22 to house the protruding elements 42 and to receive the driver tool 90 (one embodiment of which is shown in FIG. 9) that deploys and retracts the protruding elements 42.

As shown in FIG. 2, one embodiment of protruding elements 42 has a curved body 44 that is received in a side channel 46 of shaft 22. Side channel 46 is shaped to correspond to the curved body 44 of protruding element 42. Each protruding element 42 also features a grasping area 48 that is preferably pointed or otherwise shaped to securely engage and secure fixation element 20 in bone. In some embodiments, curved body 44 of protruding element 42 has a series of ratchet teeth 50 that are adapted to cooperate with a driver to deploy or retract protruding elements 42.

When deployed, protruding elements 42 extend out from openings 52 on shaft 22. Openings 52 are sized to allow curved body 44 of protruding element 42 to extend out from and retract back into shaft 22. The protruding elements 42 may be deployed back toward the osteosynthetic device 12 as shown in FIG. 2) or they may be deployed toward the bone engaging end 30 of shaft, depending upon the use and design that is desired. Deployment and retraction of protruding elements 42 is coordinated via a drive tool 90 (described further below) that is adapted to be connected to a drive connector 60 on fixation element 20. FIGS. 1 and 3 show a perspective views of protruding elements 42 in their deployed positions.

Fixation element 20 is preferably cannulated to receive a guide wire during placement and to also receive a driver tool 90. The cannulated area or opening 58 of element 20 may be smooth or threaded (as shown). Also contained within opening 58 is an internal screw 54. Internal screw 54 is one way that protruding elements 42 may be deployed and retracted. In the embodiment shown, internal screw has a notch 62 that is adapted to receive driver member 94 of tool 90 and threads 56 along its substantial length. Opening 58 is preferably also threaded, which helps facilitate the placement of internal screw 54 during manufacture of element 20 or removal or insert of internal screw 54 during use, if desired. (It should be understood the internal screw 54 may take alternate forms other than a screw, such as having sliding tracks that cooperate with corresponding tracks in opening 58, sliding notches or ratchets, or any other feature that allows it to cooperate with protruding elements 42 in order to effect their deployment.)

As shown in FIG. 9, the tool 90 for deploying and retracting protruding elements 42 has an elongated shaft 92 with a grasping handle 98 at one end and a driver member 94 at the other end. The shaft 92 has an outside diameter 96 such that it may be received by and into opening 58 and may freely turn in either rotational direction. Driver member 94 is adapted to engage notch 62 of internal screw 54, similar to the way a screwdriver is adapted to engage the head of a screw. Upon rotation of tool 90, the driver member 94 rotates internal screw 54, and tool 90 operably associates threads 56 with teeth 50 of protruding elements 42. This motion causes protruding elements 42 to deploy or to retract, depending upon the direction in which tool 90 is turned.

In certain embodiments, the protruding elements 42 or the tool 90 may have a stop for preventing the protruding elements 42 from being deployed so far that the are disengaged from fixation element 20.

The tool 90 is preferably formed from a material that is biocompatible with bone tissue and is preferably titanium, a titanium alloy, stainless steel, or a cobalt chromium alloy. It should be appreciated, however, that other materials may be used without detracting or departing from the spirit and scope of this invention. Furthermore, although one embodiment of tool 90 and its use has been described, the mechanism for deploying and retracting protruding elements 42 may be provided in many different forms without departing from scope and spirit and scope of the present invention.

An alternate embodiment of fixation element 20 and protruding elements 42 is shown in FIG. 4. This embodiment has protruding elements 42 with bendable arms 64. Bendable arms may be made of any biocompatible material, but are preferably made of nitinol or another type of biocompatible, bendable material. Arms 64 may lay flat within windows 66 of fixation element 20, and upon being deployed, they bend out and engage bone.

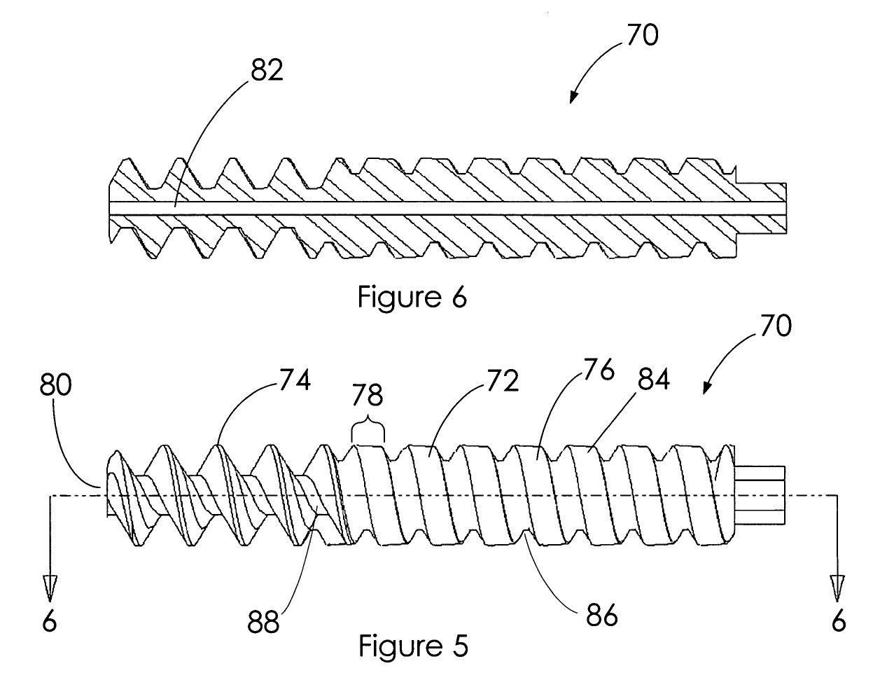

An alternate embodiment of a fixation structure 70 is shown in FIGS. 5 and 6. In this embodiment, the shaft 72 is fully threaded, although some threads are narrow threads 74 and some threads are flat threads 76 with a flat crest 78. The bone engaging end 80 of shaft 72 preferably has narrow threads 74 (or conventional bone screw threads with a narrow crest), which are adapted to engage bone and secure fixation element 70 in place. The flat threads 76 are adapted to engage bone (to the extent that the fixation element 70 is driven into bone as far down as flat threads 76 are located), but they are also adapted to slide within device 12. Flat threads 76 are smooth enough and preferably close enough together that they do not get “hung up” on the edges of hole 16 during compression.

The thread pitch (i.e., the distance between threads 74 and 76) may be between about 1 and about 5 mm, although this may be greater or smaller depending upon the size of the element 70 or the use of element in varying applications.

The distance between narrow threads 74 should be sufficient to allow threads 74 to achieve purchase into bone, but no so far apart that they weaken the integrity of element 70. The distance between flat threads 76 should also be sufficient to allow the threads 76 to achieve purchase into bone, but not so far apart that the threads 76 interfere with the ability of element 70 to slide within device 12, as shown in FIG. 8.

Flat threads 76 may also be provided with a slightly tapered crest portion 84, which may help improve the sliding of element 70 within device 12. If provided, tapered crest portion 84 may require some additional toggling during insertion of element 70, but once in place, threads 76 fall into place and allow the compression sliding to take place.

In some embodiments, the crest width for narrow threads 74 may be between about 0.1 mm and about 2 mm, although greater or smaller distances may be provided depending upon the size of element 70 and its ultimate use. Additionally, in other embodiments, the width for flat threads 76 may be between about 3 mm and about 6 mm, although greater or smaller distances may be provided depending upon the size of element 70 and its ultimate use.

In preferred embodiments, flat threads 76 are disposed along the substantial length of shaft 72. Flat threads 76 allow fixation element 70 to maintain sliding contact with device 12, but they also increase the amount of purchase that fixation element 70 may achieve, particularly in healthy bone. Flat threads 76 also reduce the amount of bone that must be removed. They allow the use of a complementary reamer that requires removal of less bone because the diameter of the screw is the same as the diameter of the hole 16—there is no need to drill a hole that compensates for the additional height of threads of prior art screws.

Fixation structure 70 has a drive connector similar to the drive connector described above. It is also provided with an opening 82 that allows it to be placed using a guide wire. Although not shown, fixation structure 70 may also have protruding elements 42 (and related channels and a threaded internal opening with an internal screw) to help facilitate the placement of element 70.

The fracture treatment system 10 may be inserted into a patient using a known closed intramedullary surgical technique, which requires minimal exposure of the femur. Generally, the intramedullary canal of the bone (e.g., a femur) is reamed with an appropriate known reaming tool to create a void for insertion of an osteosynthetic device, such as nail 12. (Progressively larger reamers may be used to increase the diameter of the void.) A guide pin or guide wire may be inserted into the reamed area, and the device 12 is guided into the reamed canal. The position of the device (including the orientation of the holes) may be verified by image intensification, such as a C-arm or x-ray.

When the rod is properly oriented, instrumentation may be used to prepare appropriate openings in the treatment area to receive fixation elements 20, 70 using known techniques. However, it is not necessary to use the separate types of drill diameters that were previously required for use of prior art screws, particularly for the use of fixation element 70. For example, prior art preparation required a hole in the femoral head and neck to be prepared with a “step-drill” or a “step-reamer” containing two diameters: a smaller diameter at its driving end corresponding to the root diameter (or minor diameter) of the lag screw thread; and a larger diameter which is equal to the diameter of the smooth portion of lag screw. This second diameter is required to provide an area in the bone that is as close as possible to the diameter of the hole of the nail but that is not too large, which required a great deal of precision. The hole should be large enough to receive the screw, but tight enough that excess bone is not removed. This preparation allowed for lag screwing the femoral head as well as sliding compression of a femoral neck fracture.

However, although step drilling is still used in connection with placing the present fixation elements 20, 70, the second diameter reamer may be decreased in size. This is primarily because the second diameter opening need only be as large as the minor diameter 86 of substantially straight flutes 24 and/or flat threads 76 so that they can achieve purchase into bone on the other side of fracture, but still allow the shaft to cooperate with opening 16. Among other benefits, this reduces the need for such great accuracy during placement of elements 20, 70. The hole that is reamed does not need to be as exact as with the prior art elements because the threads 76 and flutes 24 just need an area started to allow them to grasp bone. It is not necessary for the entire area to be pre-reamed and precisely sized.

It is also possible for element 70 to be provided with a self-cutting element 88 that will facilitate the ability of flat threads 6 to achieve purchase into bone.

Next, a driver is used to align fixation element 20, 70 with the holes 16. A guide wire may be used to determine proper position of fixation element 20, 70 in the femoral head and the fixation element 20, 70 is driven into place. The flutes 24 and/or narrow threads 74 engage bone opposite the fracture site. If provided, the substantially smooth portion 32 and/or flat threads 76 slide through holes 16. A driver may be used to compress fixation element to a desired degree. It is also possible for a compression screw to be used. If provided, compression screw should be placed using techniques known in the art. If protruding elements 24 are provided, they may be deployed using tool 90, using, for example, the various methods described above.

In some embodiments, an anchoring member may be optionally inserted through additional holes in device 12, if provided, to provide auxiliary support to proximal bone fragments. The area is reamed in an appropriate manner prior to insertion of the optional anchoring member.

In other embodiments, an optional set screw may be inserted through a hole at the top of device 12. Typically, a set screw has a tip that wedges against fixation structure to further secure it against rotation.

It will be appreciated that changes and modifications, additions and deletions may be made to the structures and methods recited above and shown in the drawings without departing from the scope or spirit of the invention and the following claims.

What is claimed is:

1. A fixation element, comprising:
(a) a shaft having a bone engaging end portion and a driving end portion;
(b) the bone engaging end portion having a series of substantially straight flutes for engaging bone;
(c) the shaft having one or more protruding elements adapted to be deployed to engage bone and to secure the fixation element in place during use;
(d) the driving end portion adapted to receive a tool for deploying or retracting the one or more protruding elements.

2. The fixation element of claim 1, further comprising a substantially smooth portion of the shaft.

3. The fixation element of claim 2, wherein the substantially smooth portion of the shaft is adapted to allow the fixation element shaft to slide within an osteosynthetic device for sliding compression of a fracture.

4. The fixation element of claim 1, wherein the substantially straight flutes comprise the substantial portion of the shaft.

5. The fixation element of claim 1, further comprising one or more side channels in the shaft that house the one or more protruding elements.

6. The fixation element of claim 1, wherein the shaft of the fixation element comprises an opening that houses an internal screw that is adapted to receive a tool for deploying or retracting the protruding elements.

7. The fixation element of claim 1, wherein the fixation element is used in connection with an intramedullary nail or a bone plate.

8. A method of treating a hip fracture, comprising:
(a) implanting an osteosynthetic device having at least one opening through the nail into the patient's femoral canal or onto the side of a patient's femur;
(b) inserting the fixation element of claim 1 into the opening of the osteosynthetic device and into the patient's femoral head, such that the fixation element crosses the fracture;
(c) deploying the one or more protruding elements of the fixation element of claim 1 to engage the femoral head and secure the fixation element from axial and rotational movement; and
(d) securing the fracture to achieve fixation.

9. A fixation element, comprising:
(a) a shaft comprising threads having a substantially flat crest along a portion of the shaft; and
(b) a bone engaging end portion comprising threads having a narrow crest for engaging bone.

10. The fixation element of claim 9, wherein the threads having a substantially flat crest are adapted to allow the shaft to slide within an osteosynthetic device for sliding compression of a fracture.

11. The fixation element of claim 9, wherein the substantially flat crest comprises a tapered crest portion.

12. A method of treating a hip fracture, comprising:
(a) implanting an osteosynthetic device having at least one opening through the nail into the patient's femoral canal on onto the side of a patient's femur;
(b) inserting the fixation element of claim 9 into the opening of the osteosynthetic device and into the patient's femoral head, such that the fixation element crosses the fracture; and
(c) securing the fracture to achieve fixation.
